(19) United States
(12) Patent Application Publication
(10) Pub. No.: US 2020/0174000 A1
(43) Pub. Date: Jun. 4, 2020

(54) MATERIALS AND METHODS FOR DETECTING AND/OR TREATING DUCTAL CARCINOMA IN SITU AND RELATED SYMPTOMS

(71) Applicants: Adrian L. Harris, Oxford (GB); Fiona Ginty, Niskayuna, NY (US); Sanghee Cho, Niskayuna, NY (US); The Trustees of Indiana University, Indianapolis, IN (US)

(72) Inventors: Sunil S Badve, Indianapolis, IN (US); Yesmin Gokmen-Polar, Noblesville, IN (US); Adrian L Harris, Oxford (GB); Fiona Ginty, Niskayuna, NY (US); Sanghee Cho, Niskayuna, NY (US)

(73) Assignee: The Trustees of Indiana University, Indianapolis, IN (US)

(21) Appl. No.: 16/700,157

(22) Filed: Dec. 2, 2019

Related U.S. Application Data
(60) Provisional application No. 62/774,653, filed on Dec. 3, 2018.

(51) Int. Cl. G01N 33/574

(52) U.S. Cl. CPC G01N 33/57488

(57) ABSTRACT

Various aspects and embodiments disclosed herein relate generally to the modelling, treatment, reducing resistance to the treatment, prevention, and diagnosis of diseases/symptoms related to ductal carcinoma in situ (DCIS). Embodiments include methods of detecting and/or treating diseases/symptoms related to ductal carcinoma in situ (DCIS), comprising the steps of: providing a sample of blood, cells, or tissue from a person suspected of having ductal carcinoma in situ; and detecting one or more epithelial markers in the sample.

This application claims priority to U.S. Ser. No. 62/774,653 filed Dec. 3, 2018, which is expressly incorporated by reference herein in its entirety.

### STATEMENT OF GOVERNMENTAL RIGHTS

This invention was made with government support under CA194600 awarded by the National Institutes of Health. The Government has certain rights in the invention.

Various aspects and embodiments disclosed herein relate generally to the modelling, treatment, reducing resistance to the treatment, prevention, and diagnosis of diseases/symptoms related to ductal carcinoma in situ (DCIS).

Ductal carcinoma in situ (DCIS) is a noninvasive breast cancer but some have shown to develop invasive breast cancer. Women having ductal carcinoma in situ (DCIS) currently undergo treatment as if they have breast cancer because it is difficult to elucidate which DCIS lesions will progress to invasive breast cancer or remain indolent. Surgery is the mainstay for the treatment of DCIS and on the clinic-pathological features, this may be followed by radiotherapy and/or endocrine therapy. The precise risk of malignant transformation from DCIS to invasive cancer is not well understood because there are relatively few case series of patients with untreated DCIS.

DCIS accounts for at least 20% of breast cancers. Assessment of cases that are likely to recur is difficult and requires multiple tissue sections for analysis of individual proteins or mRNA. Further, factors associated with recurrence of DCIS or progression to invasive carcinoma are not well delineated. Therefore, development of a new method and/or treatment is much needed.

One embodiment includes a method of detecting one or more epithelial markers in a person suspected of having recurrence of ductal carcinoma in situ, comprising: providing a sample of blood, cells, or tissue from the person suspected of having recurrence of ductal carcinoma in situ; and detecting one or more epithelial markers in the sample, wherein the one or more cellular epithelial markers comprise estrogen receptor (ER), human epidermal growth factor receptor 2 (HER2), SLC7A5 and/or cMET.

Another embodiment includes the method according to the preceeding embodiment, wherein the person has low risk of recurrence of ductal carcinoma in situ when the proportion of cellular expression of ER and/or cMET is high in the sample.

Another embodiment includes the method according to any one of the preceeding embodiments, wherein the person has increased risk of recurrence of ductal carcinoma in situ when the cellular expression of HER2 and/or SLC7A5 is high in the sample.

Another embodiment includes the method according to any one of the preceeding embodiments, further comprising staining the sample with at least one marker, quantifying cellular expression, performing k-means clustering to determine proportion of cells expressing said markers, performing regression analysis, and/or determining likelihood of recurrence by logistic regression analysis.

Another embodiment includes the method according to any one of the preceeding embodiments, wherein the step of staining comprises multiplexed immunofluorescence staining.

Another embodiment includes the method according to any one the preceeding embodiments, wherein the at least one marker comprises HER4, CK56, ABCG2, PTEN, S6, CKAE1, PR, ER, NaKATPase, CK19, ALDH1, CK PCK26, cMET, CD44v6, HER2, CDCP1, p53, CK15, COX2, VEGFR2, ABCb1, HTF9C, CD10, MRP4, CEACAM5, EGFR, p21, MRP5, SLC7A5, Ki67, and/or DAPI.

Another embodiment includes the method according to any one of the preceeding embodiments and/or calculating probability of recurrence, further comprising performing Escore analysis developed by logistic regression model and/or classification model on recurrence.

Another embodiment includes a method according to detecting one or more epithelial markers in a person suspected of having invasive carcinoma, comprising: providing a sample of blood, cells, or tissue from a person suspected of having ductal carcinoma in situ; and detecting one or more epithelial markers in the sample, wherein the one or more epithelial markers comprise estrogen receptor (ER), human epidermal growth factor receptor 2 (HER2), SLC7A5 and/or cMET.

Another embodiment includes the method according to any one of the preceeding embodiments, wherein the person has low risk of having invasive carcinoma when the expression of ER and/or cMET is high in the sample.

Another embodiment includes the method according to any one of the preceeding embodiments, wherein the person has increased risk of having invasive carcinoma when the expression of HER2 and/or SLC7A5 is high in the sample.

Another embodiment includes the method according to any one of the preceeding embodiments, further comprising staining the sample with at least one marker, quantifying cellular expression or intensity, k-means clustering to quantify proportion of cells expressing said protein, and/or determining likelihood of recurrence by performing logistic regression analysis.

Another embodiment includes the method according to any one of the preceeding embodiments, wherein the step of staining comprises multiplexed immunofluorescence staining.

Another embodiment includes the method according to any one the preceeding embodiments, wherein the at least one marker comprises HER4, CK56, ABCG2, PTEN, S6, CKAE1, PR, ER, NaKATPase, CK19, ALDH1, CK PCK26, cMET, CD44v6, HER2, CDCP1, p53, CK15, COX2, VEGFR2, ABCb1, HTF9C, CD10, MRP4, CEACAM5, EGFR, p21, MRP5, SLC7A5, Ki67, and/or DAPI.

Another embodiment includes the method according to any one of the preceeding embodiments, further comprising performing Escore analysis developed by logistic regression model and/or classification model on recurrence.

Another embodiment includes a method of treating recurrence of ductal carcinoma in situ, comprising the steps of: detecting increased expression of one or more epithelial markers in a sample of a subject suspected of having ductal carcinoma in situ, wherein the one or more epithelial markers comprise HER2 and/or SLC7A5; providing to the subject at least one treatment regimen comprising chemotherapy, radiation therapy, endocrine therapy, breast-conserving surgery (lumpectomy), breast-removing surgery (mastectomy), and/or at least one therapeutically effective dose of a compound that inhibits the epithelial expression of HER2 and/or SLC7A5.

Another embodiment includes the method according to any one of the preceeding embodiments, wherein the compound that inhibits the epithelial expression of HER2 and/or SLC7A5 comprises a pharmaceutically acceptable salt or a metabolite thereof.

Another embodiment includes the method according to any one of the preceeding embodiments, wherein the subject is diagnosed with ductal carcinoma in situ or a similar condition.

Another embodiment includes the method according to any one of the preceeding embodiments, wherein the subject comprises an animal, a human, a cell, and/or a tissue.

Another embodiment includes a method of treating invasive carcinoma, comprising the steps of: detecting increased expression of one or more epithelial markers in a sample of a subject suspected of having ductal carcinoma in situ, wherein the one or more epithelial markers comprise HER2 and/or SLC7A5; providing to the subject at least one treatment regimen comprising chemotherapy, radiation therapy, endocrine therapy, breast-conserving surgery (lumpectomy), breast-removing surgery (mastectomy), and/or at least one therapeutically effective dose of a compound that inhibits the epithelial expression of HER2 and/or SLC7A5.

Another embodiment includes the method according to any one of the preceeding embodiments, wherein the compound that inhibits the epithelial expression of HER2 and/or SLC7A5 comprises a pharmaceutically acceptable salt or a metabolite thereof.

Another embodiment includes the method according to any one of the preceeding embodiments, wherein the subject is diagnosed with ductal carcinoma in situ or a similar condition.

Another embodiment includes the method according to any one of the preceeding embodiments, wherein the subject comprises an animal, a human, a cell, and/or a tissue.

Another embodiment includes the method according to any one of the preceeding embodiments, wherein the invasive carcinoma comprises invasive ductal carcinoma (IDC), infiltrating ductal carcinoma, invasive lobular carcinoma (ILC), adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma, and/or mixed carcinoma having features of both invasive ductal and lobular.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart documenting the number of patient samples, field of views (FOVs) and cells analyzed for the 33 markers.

FIGS. 2A-2C are matching digitally generated H&E (virtual H&E), Immuno-Fluorescence images of DCIS and graphical representation of cells in a cluster 2 dominant sample (high ER, low cMet, low Her2, low SLC7A5 expression), based on the spatial coordinates of cell clusters in that sample. Cluster 2 Dominant (ER⁺ cMET⁻).

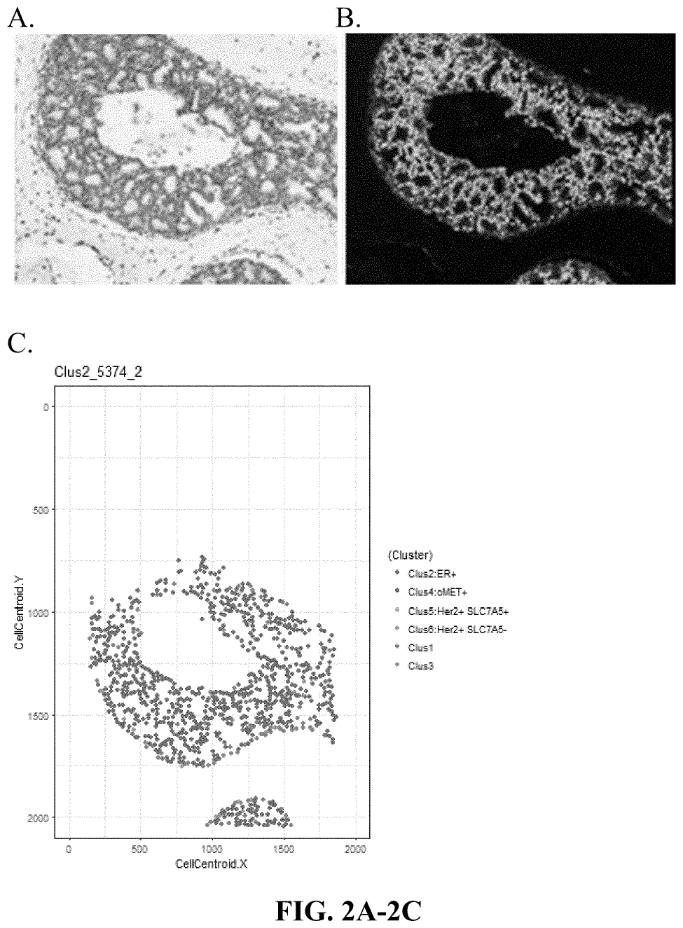

FIGS. 3A-3C are matching digitally generated H&E (virtual H&E), Immuno-Fluorescence images of DCIS and graphical representation of cells in a cluster 4 dominant sample (high ER, high cMet expression, low Her2, low SLC7A5), based on the spatial coordinates of cell clusters in that sample. Cluster 4 Dominant (ER⁺ cMET⁺).

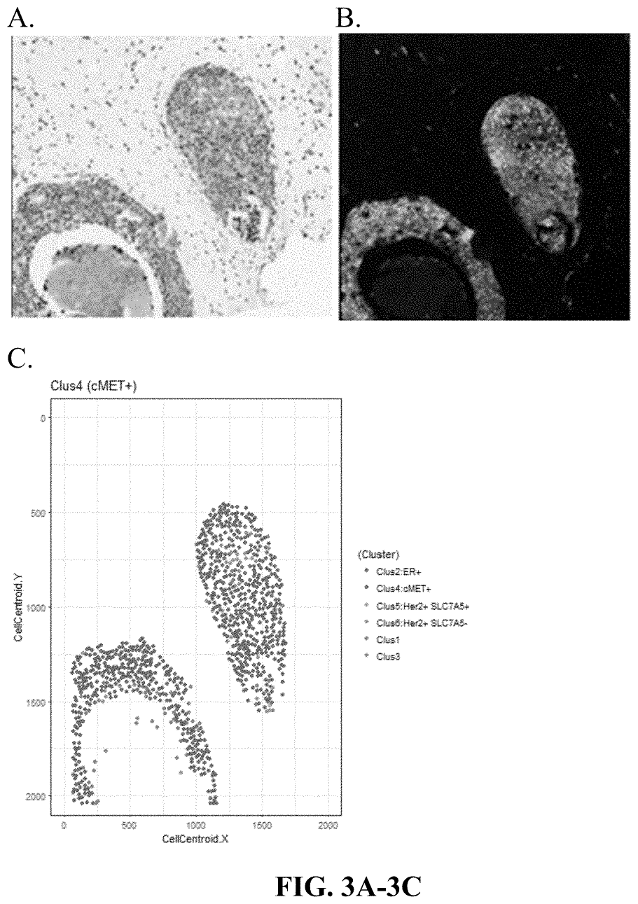

FIGS. 4A-4C are matching digitally generated H&E (virtual H&E), Immuno-Fluorescence images of DCIS and graphical representation of cells in a cluster 5 dominant sample (high Her2, high SLC7A5, low ER, medium/low cMET expression), based on the spatial coordinates of cell clusters in that sample. Cluster 5 Dominant (Her2⁺ SLC7A5⁺).

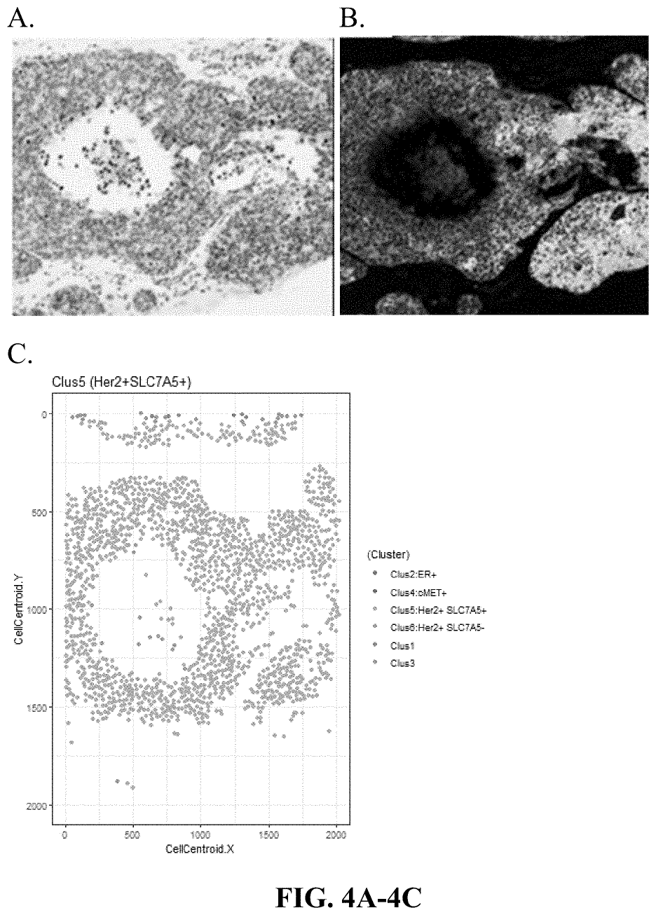

FIGS. 5A-5C are matching digitally generated H&E (virtual H&E), Immuno-Fluorescence images of DCIS and graphical representation of cells in a cluster 6 dominant sample (high Her2, low SLC7A5, low ER, low cMET expression), based on the spatial coordinates of cell clusters in that sample. Cluster 6 Dominant (Her2⁺ SCLA5⁻).

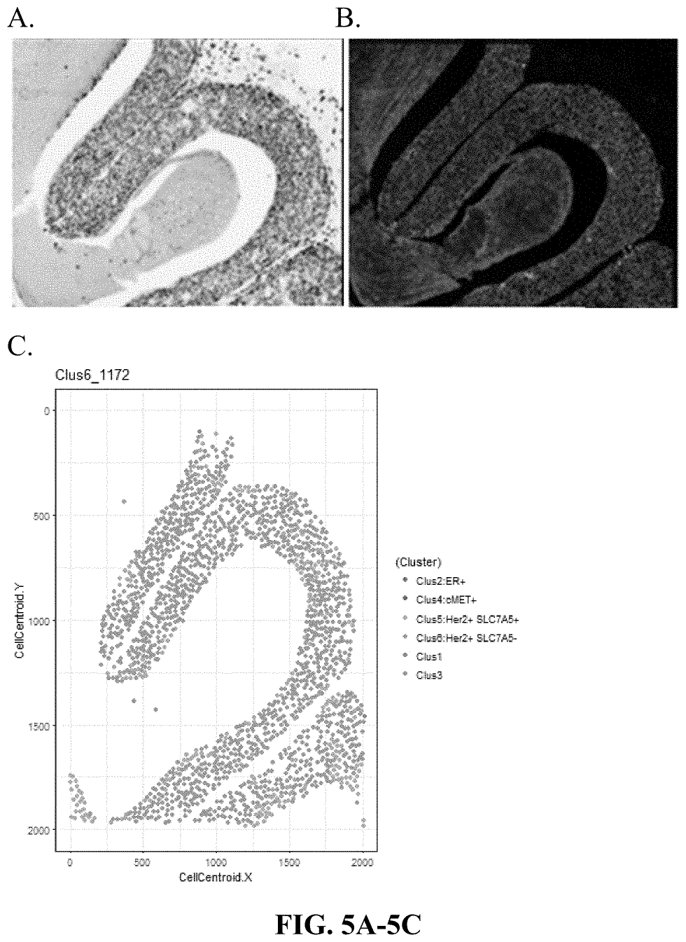

FIGS. 6A-6C are matching digitally generated H&E (virtual H&E), Immuno-Fluorescence images of DCIS and graphical representation of cells in a sample with a mixture of clusters 2 and 4 (high ER, low Her2, low SLC7A5, mixed (low and high) cMET expression), based on the spatial coordinates of cell clusters in that sample. Cluster 2 and 4 Dominant (ER⁺ cMET^{+/-}).

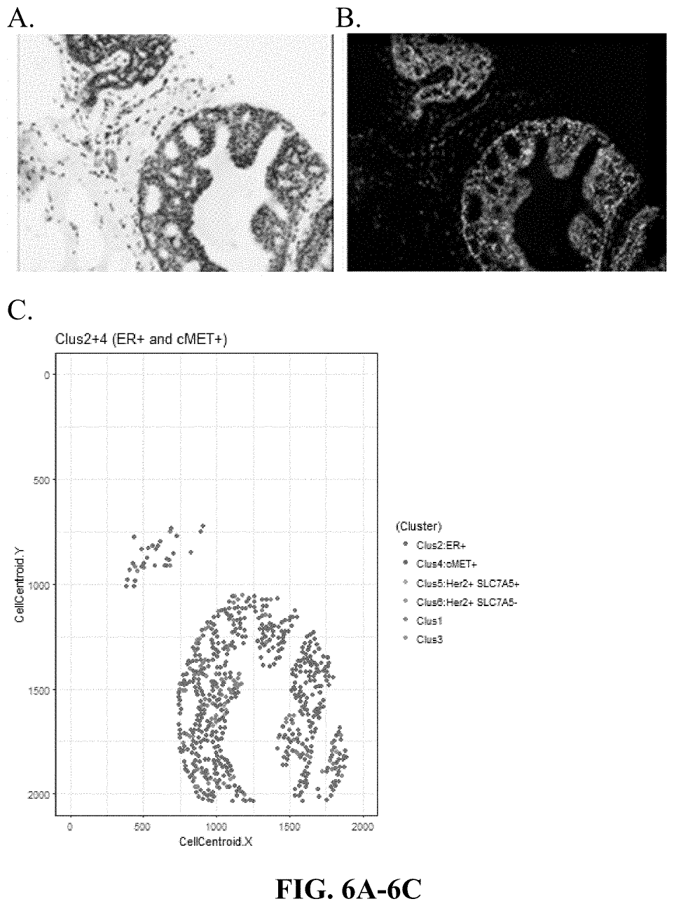

FIGS. 7A-7C are matching digitally generated H&E (virtual H&E), Immuno-Fluorescence images of DCIS and graphical representation of cells in a sample with a mixture of clusters 5 and 6 (high Her2, low ER, mixed (low and high) SLC7A5, low cMET expression), based on the spatial coordinates of cell clusters in that sample. Cluster 5 and 6 Dominant (Her2⁺ SCLA5^{+/-}).

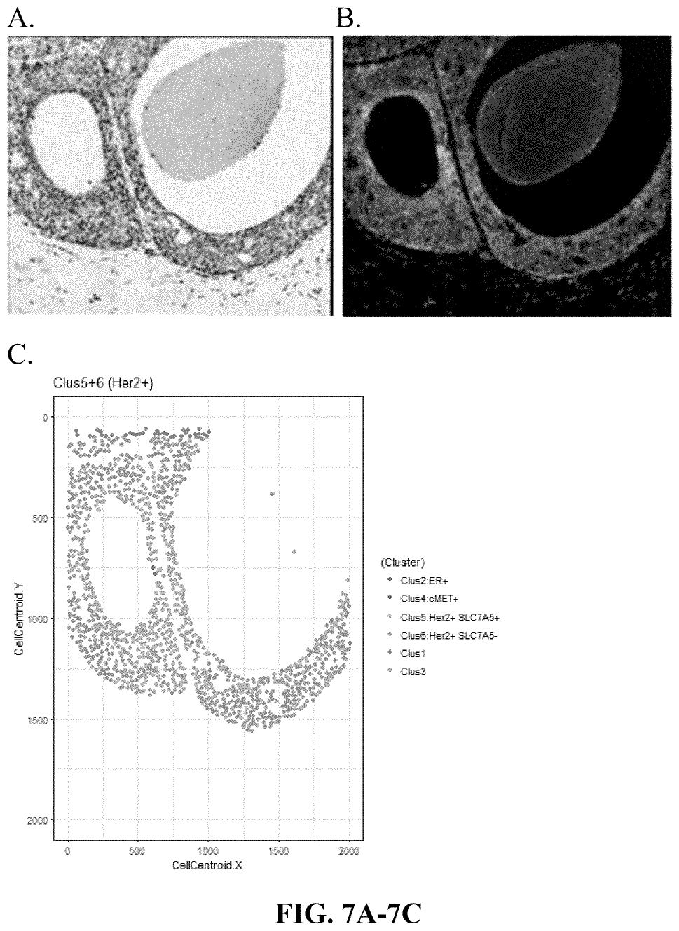

FIGS. 8A-8C are matching digitally generated H&E (virtual H&E), Immuno-Fluorescence images of DCIS and graphical representation of cells in a sample with a heterogeneous mixture of clusters 1-6, based on the spatial coordinates of cell clusters in that sample. Heterogeneous Mixture of Clusters 1-6.

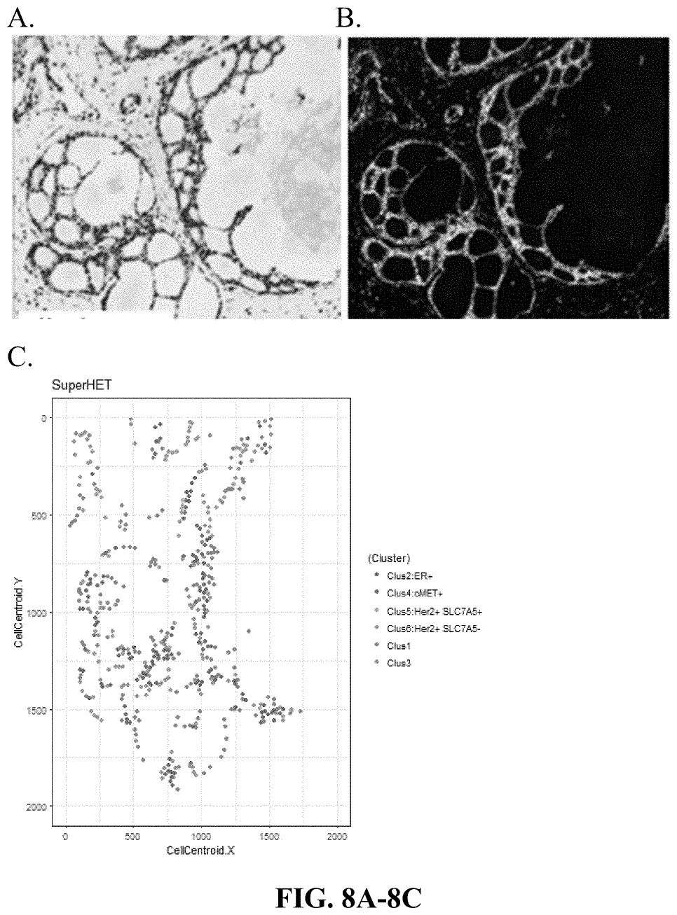

FIGS. 9A-9C are matching digitally generated H&E (virtual H&E), Immuno-Fluorescence images of DCIS and graphical representation of cells in a sample with a mixture of clusters 4 and 5 (Mixed ER, mixed Her2, mixed SLC7A5, mixed cMET) based on the spatial coordinates of cell clusters in that sample. Clusters 4 and 5 Dominant (Her2⁺ ER⁺ cMET⁺ SCLA5⁺).

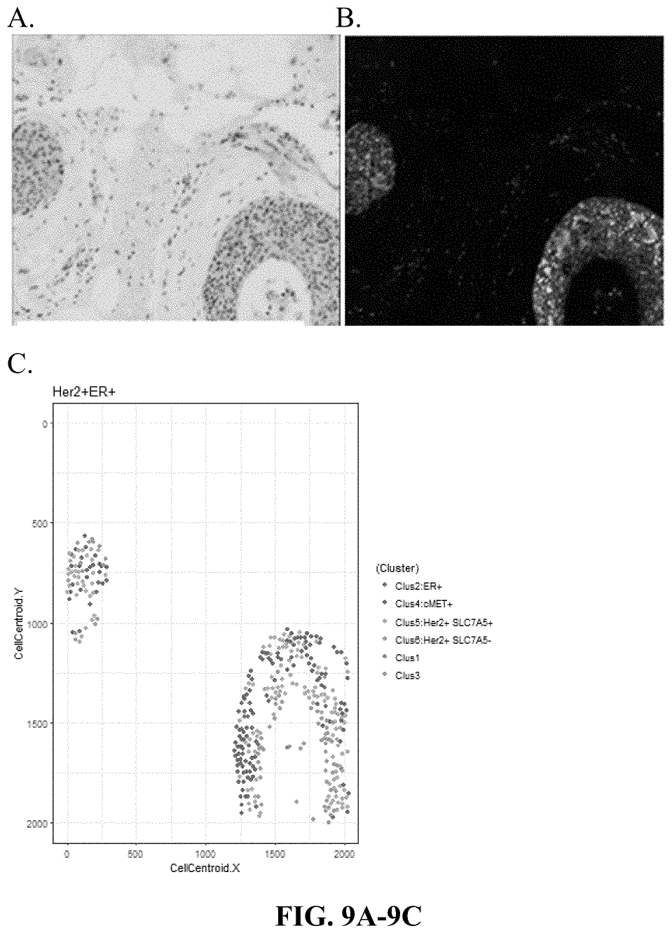

FIG. 10 is a heatmap from k-means analysis of cell expression of 4 markers: Her2, SLC7A5, ER, and cMET resulting in 6 groups or clusters. Cluster 2 shows ER positive cells that are negative for Her2, SLC7A5 and cMET, and cluster 4 shows cMET positive cells that are generally positive for ER and negative for Her2 and SLC7A5. Two clusters (clusters 5 and 6) are positive for Her2 and cluster 5 is also positive for SLC7A5 and negative for ER and cMET. Heat map of single cell-based clusters for Her2, SLC7A5, ER and Her2, and percent of those clusters in recurring (Y) and non-recurring patients (N) and p-values for differences.

FIG. 11 shows distribution of clusters 2, 4, 5 and 6 in recurring and non-recurring patients. Recurrent patients appear to have very little cluster 2 and 4 type cells and more cluster 5 and 6 type cells.

FIG. 12 shows development of logistic regression-based algorithm for identifying likelihood of recurrence. Classification using logistic regression with two input variables gives AUC of 0.79 with TPR=77%, TNR=79%, error rate=21.6%. Two input variables are used in the logistic regression model; one is combination of clusters 2 and 4, and the other is a combination of clusters 5 and 6. classification model used the following formula (Escore) to predict recurrence: 1.77*(% Clus5&6)−2.78*(% Clus2&4)>13.

FIG. 13 is a Kaplan Meier plot illustrating the impact of the Escore on identifying the likelihood of recurrence. Kaplan-Meier plot of recurring and non-recurring patients based on E-score generated from the classification model 1.77*(% Clus5&6)−2.78*(% Clus2&4)>13.

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

A low risk of recurrence has a risk level defined by a low Escore: 1.77*(% Clus5&6)−2.78*(% Clus2&4)<13. An expression of ER and/or cMET that is high in the sample refers to a patient with high % Clus2&4 (in the Escore equation), the level of cMET and/or ER in the cells and refers to high proportion of those cells in the sample.

As used herein, unless explicitly stated otherwise or clearly implied otherwise the term ‘about’ refers to a range of values plus or minus 10 percent, e.g. about 1.0 encompasses values from 0.9 to 1.1.

The term, “treating” as used herein unless stated or implied otherwise, includes administering to a human or an animal patient at least one dose of a compound, treating includes preventing or lessening the likelihood and/or severity of at least one disease as well as limiting the length of an illness or the severity of an illness, treating may or may not result in a cure of the disease.

As used herein, unless explicitly stated otherwise or clearly implied otherwise the terms ‘therapeutically effective dose,’ ‘therapeutically effective amounts,’ and the like, refer to a portion of a compound that has a net positive effect on health and well being of a human or other animal. Therapeutic effects may include an improvement in longevity, quality of life and the like these effects also may also include a reduced susceptibility to developing disease or deteriorating health or well being. The effects may be immediately realized after a single dose and/or treatment or they may be cumulative realized after a series of doses and/or treatments. A “therapeutically effective amount” in general means the amount that, when administered to a subject or animal for treating a disease, is sufficient to affect the desired degree of treatment for the disease.

As used herein, “inhibition” or “inhibitory activity” each encompass whole or partial reduction of activity or effect of an enzyme or all and/or part of a pathway that includes an enzyme that is effected either directly or indirectly by the inhibitor or a pathway that is effected either directly or indirectly by the activity of the enzyme which is effected either directly or indirectly by the inhibitor.

As used herein, “breast cancer” can include, but is not limited to, ductal carcinoma in situ (DCIS), invasive breast cancer, inflammatory breast cancer, angiosarcoma of the breast, and/or paget disease of the nipple.

As used herein, “invasive carcinoma” or “invasive breast cancer” refers to a type of cancer that can include, but is not limited to, invasive ductal carcinoma (IDC), infiltrating ductal carcinoma, invasive lobular carcinoma (ILC), adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma, and/or mixed carcinoma having features of both invasive ductal and lobular.

As used herein, the term “pharmaceutically acceptable salt” is defined as a salt wherein the desired biological activity of the inhibitor is maintained and which exhibits a minimum of undesired toxicological effects. Non-limiting examples of such a salt are (a) acid addition salts formed with inorganic acids (e.g., hydrochloric acid, hydrobromic acid, sulphuric acid, phosphoric acid, nitric acid, and the like), and salts formed with organic acids (such as e.g. acetic acid, oxalic acid, tartaric acid, succinic acid, malic acid, ascorbic acid, benzoic acid, tannic acid, palmitic acid, polyglutamic acid, naphthalene sulphonic acid, naphthalene disulphonic acid, polygalacturonic acid and the like); (b) base additional salts formed with metal cations such as zinc, calcium, bismuth, barium, magnesium, aluminum, copper, cobalt, nickel, cadmium, sodium, potassium and the like, or with a cation formed from ammonia, N,N-dibenzylethylenediamine, D-glucosamine, tetraethylammonium or ethylenediamine; or (c) combinations of (a) and (b); e.g. a zinc tannate or the like.

Pharmaceutically acceptable salts include salts of compounds of the invention that are safe and effective for use in mammals and that possess a desired therapeutic activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention may form pharmaceutically acceptable salts with various amino acids. Suitable base salts include, but are not limited to, aluminum, calcium, lithium, magnesium, potassium, sodium, zinc, and diethanolamine salts. For additional information on some pharmaceutically acceptable salts that can be used to practice the invention. See, e.g., reviews such as Berge, et al., 66 J. PHARM. SCI. 1-19 (1977), Haynes, et al, J. Pharma. Sci., Vol. 94, No. 10, October 2005, pgs. 2111-2120 and See, e.g., P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol. 66, No. 1, January 1977.

Pharmaceutical formulation: The compounds of the invention and their salts may be formulated as pharmaceutical compositions for administration. Such pharmaceutical compositions and processes for making the same are known in the art for both humans and non-human mammals. See, e.g., REMINGTON: THE SCIENCE AND PRACTICE OF PHARMACY, (A. Gennaro, et al., eds., 19^{th} ed., Mack Publishing Co., 1995). Formulations can be administered through various means, including oral administration, parenteral administration such as injection (intramuscular, subcutaneous, intravenous, intraperitoneal) or the like; transdermal administration such as dipping, spray, bathing, washing, pouring-on and spotting-on, and dusting, or the like. Additional active ingredients may be included in the formulation containing a compound of the invention or a salt thereof.

The pharmaceutical formulations of the present invention include those suitable for oral, parenteral (including subcutaneous, intradermal, intramuscular and intravenous) and rectal administration. The formulations may be presented in unit dosage form and may be prepared by any of the methods well known in the art of pharmacy. All methods include the step of bringing into association the active ingredient, i.e., the compound or salt of the present invention, with the carrier. In general, the formulations are prepared by uniformly and intimately bringing into association the active ingredient with a liquid carrier or, a finely divided solid carrier or both, and then, if necessary, forming the associated mixture into the desired formulation.

The pharmaceutical formulations of the present invention suitable for oral administration may be presented as discrete units, such as a capsule, cachet, tablet, or lozenge, each containing a predetermined amount of the active ingredient; as a powder or granules; as a solution or a suspension in an aqueous liquid or non-aqueous liquid such as a syrup, elixir or a draught, or as an oil-in-water liquid emulsion or a water-in-oil liquid emulsion. The formulation may also be a bolus, electuary or paste.

The pharmaceutical formulations of the present invention suitable for parenteral administration include aqueous and non-aqueous sterile injection solutions, and may also include an antioxidant, buffer, a bacteriostat and a solution which renders the composition isotonic with the blood of the recipient, and aqueous and non-aqueous sterile suspensions which may contain, for example, a suspending agent and a thickening agent. The formulations may be presented in a single unit-dose or multi-dose containers and may be stored in a lyophilized condition requiring the addition of a sterile liquid carrier prior to use.

Pharmaceutically acceptable carrier: Pharmaceutically acceptable carrier, unless stated or implied otherwise, is used herein to describe any ingredient other than the active component(s) that maybe included in a formulation. The choice of carrier will to a large extent depend on factors such as the particular mode of administration, the effect of the carrier on solubility and stability, and the nature of the dosage form.

A tablet may be made by compressing or moulding the active ingredient with the pharmaceutically acceptable carrier. Compressed tablets may be prepared by compressing in a suitable machine the active ingredient in a free-flowing form, such as a powder or granules, in admixture with, for example, a binding agent, an inert diluent, a lubricating agent, a disintegrating and/or a surface active agent. Moulded tablets may be prepared by moulding in a suitable machine a mixture of the powdered active ingredient moistened with an inert liquid diluent. The tablets may optionally be coated or scored and may be formulated so as to provide slow or controlled release of the active ingredient.

The successful implementation of the breast screening programs in developed countries have resulted in the identification of a large number of putative precursor lesions of invasive carcinoma. Ductal carcinoma in situ (DCIS) is a non-obligate precursor lesion that is managed aggressively. Most patients get treated with surgery followed by post-operative radiation therapy. These have been documented to decrease the incidence of recurrence and development of invasive cancer. In addition, the UK/ANZ DCIS (UK, Australia, and New Zealand Ductal Carcinoma in situ) trial and the NSABP (National Surgical Adjuvant Breast and Bowel Project) B-24 clinical trials further demonstrated a significant reduction in frequency of DCIS recurrence by the addition of endocrine therapy with resultant recurrence rates below 10%.

DCIS, if left untreated, will not progress to invasive carcinoma in around 20-50% of patients. This has led to significant concerns regarding overtreatment of patients. Currently, there are many trials that are enrolling patients with DCIS for non-surgical management based on histological features of DCIS. Low Risk DCIS cases are being enrolled in the LORIS trial in the United Kingdom and LORD (Low Risk DCIS) trial in Europe for non-surgical management by active surveillance. This is similar to the COMET (Comparing Operative to Monitoring and Endocrine Therapy for low risk DCIS) trial in the USA. The presence of comedo-necrosis is an important exclusion criterion, however, histological features are subjective and there is poor inter-observer agreement. A recent survey of more than 30 international recognized breast pathologists documented marked variability in definition of comedo-necrosis. This subjectively will significantly impact on patient enrollment and final study results. There is a clear need for better understanding the biology of DCIS and the pathways leading to (or associated with) progression.

A number of tools have been used for the prognostication of DCIS. These include histological features, and single as well as panels of immunohistochemistry assays, in addition to multiplex PCR for mRNAs. Analysis of the 12-year follow-up data of the Eastern Cooperative Oncology Group (ECOG) E 5194 trial has confirmed the role of histological features (high grade) in predicting likelihood of recurrence. This was also confirmed in analysis of 57,222 DCIS cases from the SEER (The Surveillance, Epidemiology, and End Results) database previously. The expression of estrogen and progesterone receptors (ER and PR) is also associated with decreased risk of recurrence. In contrast, proliferation markers such as Ki67 are associated with a higher risk. Some studies have analyzed the expression of p16, COX2 and Ki67 to identify an IHC based predictor for the likelihood of recurrence. In a multivariable model, DCIS lesions that were p16⁺/COX2⁺/Ki67⁺ or those detected by palpation were statistically significantly associated with subsequent invasive cancer. Based on these initial analyses, they have identified a panel of IHC biomarkers (PR, HER2, Ki67, COX2, p16/INK4A, FOXA1 and SIAH2), which is commercially available through Prelude's CLIA-approved lab as DCISION RT™ (Decision score (DS)). In multivariable analysis, DS, but not nuclear grade, correlated with benefit of radiotherapy in the SweDCIS cohort (Warnberg SABCS 2017).

In this disclosure, it was designed to further understand the impact of a large number of biomarkers associated with recurrence/progression of DCIS using a cohort of well annotated DCIS cases with follow-up data. DAPI was used to identify the nuclei and together with NaKATPase, pan-cytokeratin and S6 used for epithelial cell segmentation. The utility of ER, PR, HER2, Ki67, p53, COX2, and CD10, in DCIS has been well described. The HER pathway was further investigated by analyzing EGFR, HER4 and PTEN. Also investigated cancer stem cell markers (ALDH1 and CD44v6), and proteins implicated in progression (p21, VEGFR2, cMET, CDCP1, HTF9C/TRMT2A, and CEACAM5) and resistance to therapy (ABCB1, ABCG2, MRP4, MRP5, SLC7A5), in breast cancer. The GE CELL DIVE™ technology was used to analyze the cellular (co-)expression of these markers in a single FFPE section.

Further, a tissue microarray (TMA)-based cohort of DCIS cases with or without recurrence was obtained from Oxford University. Recurrence in this cohort was defined as ipsilateral DCIS, ipsilateral invasive, contralateral invasive and metastatic. Analysis for epithelial markers (e.g., HER4, CK56, ABCG2, PTEN, S6, CKAE1, PR, ER, NaKATPase, CK19, ALDH1, CK PCK26, cMET, CD44v6, HER2, CDCP1, p53, CK15, COX2, VEGFR2, ABCb1, HTF9C, CD10, MRP4, CEACAM5, EGFR, p21, MRP5, SLC7A5, Ki67, DAPI) was performed on a single 5 um formalin-fixed paraffin-embedded (FFPE) TMA section containing cases of DCIS. Briefly, FFPE sections from TMAs containing DCIS were sequentially stained and imaged for the markers. Each cycle entailed staining with 2-3 markers followed by imaging, dye inactivation, and re-staining. DAPI was used for nuclear demarcation and for registration of the images, while S6, pan-cadherin, Na⁺K⁺ ATPase and pan-cytokeratin were used for epithelial cell segmentation and quantification of each of the biomarkers in each cell. K-means clustering of cell biomarker expression, followed by logistic regression analysis was performed to identify inter-relationships between markers and association with likelihood of recurrence. Log-rank analysis was performed and the relapse-free survival data depicted using Kaplan Meier plots (KM plots). Escore was developed by logistic regression classification model Materials and Methods

Antibody Screening and Selection.

All antibodies were validated per protocol described previously. Where possible, antibodies used in clinical IHC lab were included in the screenings. After selection, each antibody was conjugated with either Cy3, Cy5 or Cy7 bis-NETS-ester dyes using standard protocols as previously described. See Gerdes et al., 2013, Highly multiplexed single-cell analysis of formalin-fixed, paraffin-embedded cancer tissue. PNAS, 110:(29):11982-11987.

Clinical Cohort.

De-identified DCIS cases were selected from the archives at Oxford University/Radcliffe General Hospital. The criteria for the selection were as follows 1) they were excision specimens; 2) patients did not have invasive cancer; 3) patients did not have any prior therapy. Also, additional data filtering criteria were applied during the data analysis which will be discussed in the later sections. The patients had a median follow uptime of 8 years, range 1-17 years, and were diagnosed between 1986 and 1999, to allow the long natural history of DCIS biology to be observed. Patients were primarily Caucasian, with a few Indian and Pakistani women with age range of 32-75 (median age 55). Recurrence was defined as any event in ipsilateral or contralateral breast and used as the primary endpoint. Breast cancer screening was introduced into the UK in 1988, age 50-70, so many patients in this study presented symptomatically, and were treated with radical surgery (80%), adjuvant hormone therapy (56%), and adjuvant radiotherapy (74%) and 16% had both radiotherapy and endocrine therapy [Tamoxifen].

An initial histopathological review was performed to confirm the diagnosis of DCIS. The diagnosis of DCIS was confirmed independently by two pathologists. After the review of H&Es, tissue microarrays (core diameter 2 mm) were constructed from 270 samples comprising 200 independent single cores, 2 mm dimension with 40 cores per slide and 7 slides total, plus two control arrays. IRB permissions were obtained from Oxford University (for the entire study) and waiver of IRB from Indiana University.

Using fluorescent staining of DAPI and autofluorescent image, a virtual H&E digital image was generated from each core and this was used to study the relationships between the epithelial and stromal components and to quantify the different elements (see below) within any given field. All possible fields were quantified from each case.

Multiplexed Immunofluorescence CELL DIVE™.

Multiplexed immunofluorescence staining was performed as previously described. Briefly, slides were rehydrated, underwent a two-step antigen retrieval and were stained using a Leica Bond autostainer. All the sections were analyzed for 33 markers using antibodies at concentrations as listed in Table 1. Briefly, the 33 markers and staining rounds were as follows: Round 1: CK5/6, Her4; Round 2: ABCG2, PTEN, S6; Round 3: CD20, S6, CKAE1; Round 4: PR, ER, NaKATPAse; Round 5: CK19, ALDH1, PCK26; Round 6: CD4, cMET; Round 7: CD44v6, Her2; Round 8: CDCP1, p53; Round 9: CK15, Cox2; Round 10: VEGFR2, ABCB1; Round 11: HTF9c, CD10; Round 12: MRP4, SLC7A5; Round 13: EGFR, p21; Round 14: MRP5, CEACAM5; Round 15 Ki67 (note that in total, 7 background imaging rounds were also included).

TABLE 1
Multiplexed Immunofluorescence Staining.

| | Antibody- | Staining | | Antibody- | Staining | | Antibody- | Staining | |
|---|---|---|---|---|---|---|---|---|---|
| Step | Cy3 | Conc. | | Cy5 | Conc. | | Cy7 | Conc. | |
| 0 | Background | | | | | | | | |
| 1 | Her4 | 4 | g/ml | CK 5/6 | 10 | μg/mL | | | |
| 2 | Background | | | | | | | | |
| 3 | ABCG2 | 10 | μg/mL | PTEN | 10 | μg/mL | S6 | 10 | μg/mL |
| 4 | Background | | | | | | | | |
| 5 | CD20 | 2.5 | μg/mL | S6 | 5 | μg/mL | CK AE1 | 2.5 | μg/mL |
| 6 | Background | | | | | | | | |
| 7 | MR | 10 | μg/mL | ER | 10 | μg/mL | NaKATPase | 5 | μg/mL |
| 8 | Background | | | | | | | | |
| 9 | CK19 | 5 | μg/mL | ALDH1 | 10 | μg/mL | CK PCK26 | 2.5 | μg/mL |
| 10 | Background | | | | | | | | |
| 11 | CD4 | 10 | μg/mL | C-Met | 15 | μg/mL | | | |
| 12 | CD44v6 | 5 | μg/mL | Her2 | 5 | μg/mL | | | |
| 13 | CDCP1 | 20 | μg/mL | p53 | 1 | μg/mL | | | |
| 14 | CK15 | 5 | μg/mL | Cox-2 | 20 | μg/mL | | | |
| 15 | VEGFR2 | 10 | μg/mL | MDR1/ABCB1 | 5 | μg/mL | | | |
| 16 | Background | | | | | | | | |
| 17 | HTF9C | 5 | μg/mL | CD10 | 10 | μg/mL | | | |
| 18 | MRP4 | 2.5 | μg/mL | SLC7A5 | 5 | μg/mL | | | |
| 19 | EGFR | 1 | μg/mL | p21 | 5 | μg/mL | | | |
| 20 | MRP5 | 5 | μg/mL | CEACAM5 | 2.5 | μg/mL | | | |
| 21 | | | | Ki67 | 10 | μg/mL | | | |

All fields of view (FOV) acquired were subsequently re-evaluated by a pathologist for tumor content and fields entirely composed of DCIS were further evaluated.

Image Processing and Statistical Analyses.

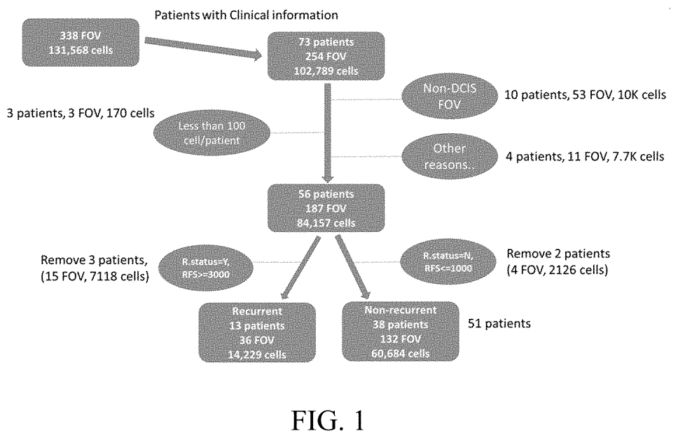

The complete image set was then reviewed after for tissue quality (tissue loss or damage) and image analysis segmentation quality. Image not passing criteria for good quality staining or minimum number of cells or histology were excluded from data analysis. Data preprocessing and single cell analysis is then done to segment cells in the epithelial and stromal compartments using the DAPI, pan-cytokeratin, S6, and NaKATPase as previously described. See Gerdes et al. 2013. Data from the multiple rounds of imaging was overlaid and tissue segmentation algorithms were applied as previously described to separate the epithelial cells and stromal compartments and biomarkers were quantified at single cell level. Several quality control steps were conducted: manual scoring of tissue quality and segmentation for every core image; 1) cell filtering based on the following criteria: epithelial cells required to have 1-2 number of nuclei; 2) each sub-cellular compartment (nucleus, membrane, cytoplasm) area>10 pixels<1500 pixels; and 3) cells in each round of staining have to have good alignment (minimum 100%) with first round of staining (automatic tissue quality index=1 at each round, which is the correlation between each image and the DAPI image). After the Quality control, data is further processed including exposure time correction, standardization to remove the batch effect, and log 2 transformation to handle the skewness of the marker intensities. Detailed flowchart of this process and patient attrition is shown in FIG. 1. Cell level intensity is represented by median within nucleus for nuclear markers (ER, PR, p21, Ki67), and median of the whole cell for rest of the markers.

The primary clinical endpoint for the purpose of univariate and multivariate analysis is (any) recurrence. Additional patient level filtering was also applied as shown in FIG. 1. First, fields of views (FOVs) were filtered if there was not enough DCIS content (% DCIS/(% DCIS+% Normal)<0.5), and patients with less than 100 cells were excluded. Patients who were reported as having no-recurrence but had too short history (<=1000 days) and who were reported as having recurrence, but with very long time to event (>=3000 days) are excluded. In univariate analysis, violin plots of the two group and recurrence/non-recurrence were compared, and t-test was performed to evaluate the mean difference. Mean of the median cell intensities per marker were used for patient level aggregation. Correlation plots/statistical test were further evaluated to examine if certain pairs of markers are correlated as a multivariate analysis.

After the above data cleaning process, extreme values (1% on both tails) are capped and standardized with zero mean and single standard deviation to remove unit effect of each marker. Then unsupervised k-means clustering is applied with number of groups k=2, 3, 4, 5, 6, 7, 8, 9, 10, 11, 12, 13, 14, and/or 15. Multiple metrics and visualizations including consensus clustering are used to determine the best number of groups to represent the data. Consensus clustering is repeating k-means clustering in a subset of the data and measuring how consistently data separates into groups. PAC (proportion of ambiguously clustered) and visual check of the heatmap determined 6 clusters were best for this set of markers. Patient profile, the proportion of cells for each clustering group, was determined for each patient.

After determining the best set of clusters, the unsupervised clustering results were related to outcome—recurrence. The correlation between patient profile and outcome is evaluated in both univariate and multivariate manner. Furthermore, a model predicting the outcome was developed based on the patient profile using logistic regression analysis. Results of the model were expressed as score (Escore) to help quantify the risk of recurrence. Leave-one-out cross validation was also performed to confirm the results. Log rank test and Kaplan Meier plot (KM plot) were generated to evaluate the effectiveness of the score.

### Examples

Referring now to FIG. 1, the workflow analysis for the DCIS TMAs is shown. Briefly, of the approximately 2 million cells analyzed, 40% were classified as epithelial and further filtering resulted final analysis of 74913 nucleated epithelial cells. (131,568 cells were included in the clustering analysis, and 75K for patient level analysis (outcome analysis). Approximately 30,000 cells did not have clinical information and others were excluded from additional criteria mentioned in above section).

The follow-up period for the patients ranged from 306 days to 6,234 days. The final cohort used in outcome analysis consisted of 13 patients with recurrence/progression of DCIS in 3000 days and 38 patients with DCIS who did not have recurrence or progression for more than 1000 days. The mean duration of follow-up for patients with recurrence was 3 years while that for patients without recurrence was 10 years. Clinical features such as patient age and menopause status were not associated with recurrence. Similarly, histological features were not associated with recurrence.

In univariate analysis, only ER, PR, HER2 were associated with likelihood of recurrence (p-value<0.05 without multiple testing correction). EGFR was associated with recurrence (p-value<0.05) but was excluded from the analysis due to overall poor quality of staining. The remainder of the markers were as follows: ABCB1, ABCG2, ALDH1, CDCP1, CD10, CD44v6, CEACAM5, CK-15, CK-19, CK-56, CK-AE1, CK-PCK26, cMET, COX2, HER4, HTF9C, Ki67, MRP4, PTEN, MRP5, NaKATPase, p53, p21, S6, SLC7A5 and VEGFR2. More specifically, Ki67 and COX2 were not associated with recurrence (p=0.561 and p=0.851 respectively).

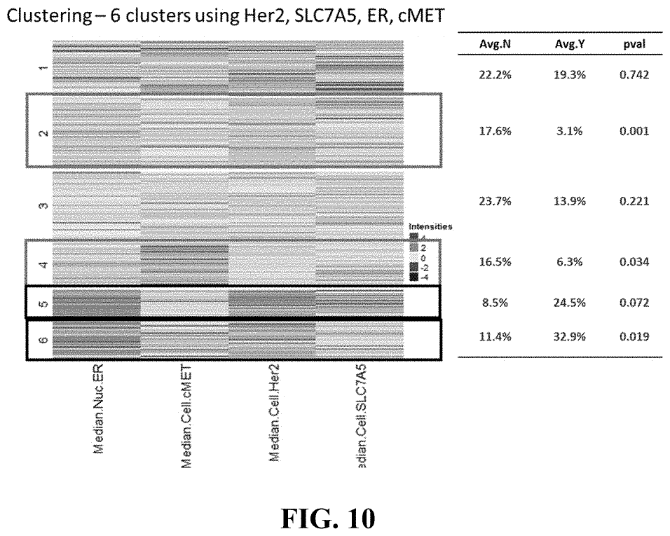

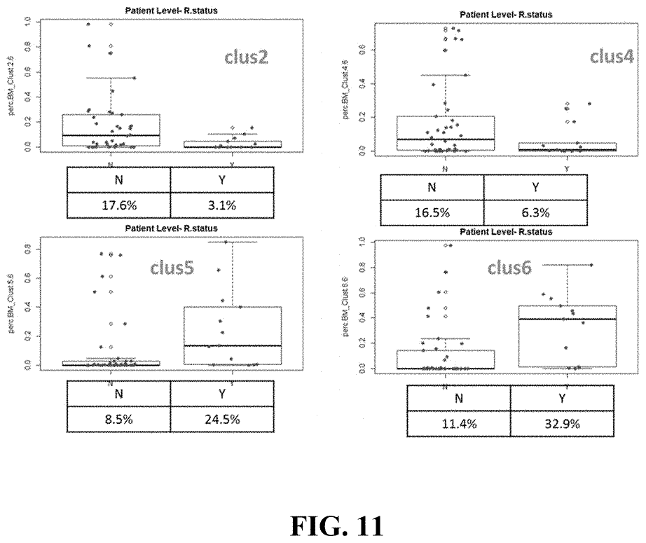

Filtering of the expression analysis by the quality, specificity, compartment localization and fields entirely composed of DCIS, in addition to availability of clinical data resulted final analysis of 31 markers in 67 cases. Correlation analyses were performed on each of the markers to identify markers that were significantly correlated in univariate analysis. K-means cluster analysis was performed using a set of 4 markers (ER, HER2, SLC7A5 and cMET) to identify 6 clusters. The pattern of expression of these 4 markers (ER, HER2, SLC7A5, and cMET) identified 6 clusters (FIG. 10) and their relationship with outcomes is shown in FIGS. 10 and 11. Cluster 2, characterized by high ER but low levels of HER2, SLC7A5 and cMET, was strongly associated with lack of recurrence (P=0.001). Similarly, cluster 4 (cMET^{high} with low levels of HER2^{low} and SLC7A5^{low}) was also associated with lack of recurrence (P=0.034). Cluster 6 (HER2^{high}, SLC7A5^{low}, and low ER) was associated with high risk of recurrence (P=0.018). Of note, High HER2 with high SLC7A5 (cluster 5) showed only a trend towards increased likelihood of recurrence (P=0.072), suggesting a possible impact of SLC7A5 in determination of recurrence.

A regression analysis-based algorithm was developed using these markers to calculate a numerical score which could predict likelihood of recurrence. As depicted in the KM plots, the HR for recurrence increases significantly (P-value 2.4E-05; p=0.02 with LOOCV) with increase in expression score (Escore).

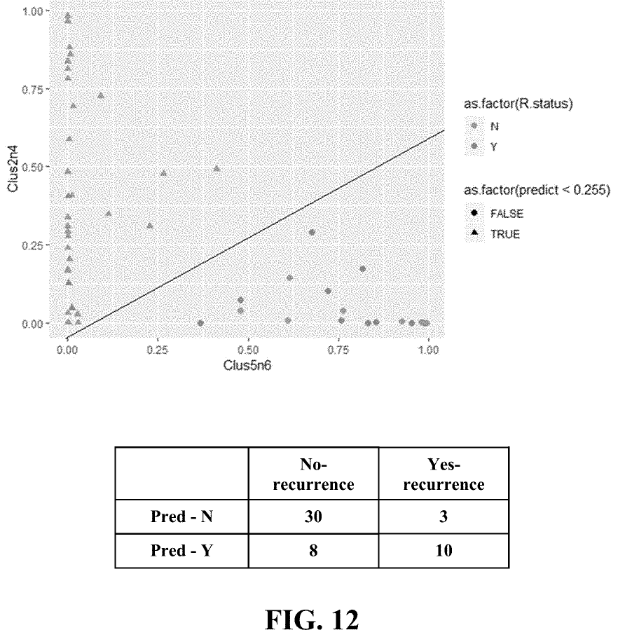

In order to further assess the clinical utility of the 4 markers, a logistic regression analysis was performed using only 2 combined cell types (clusters 2 & 4 and clusters 5 & 6). Referring now to FIG. 12, the model gave an AUC of 0.79 (0.74 with Leave-one-out cross validation) with sensitivity (TPR) of 77% and a specificity (TNR) of 79%. This analysis was further converted into an expression score, “Escore”, that predicts the likelihood of recurrence. Escore from the classification Model: 1.77*(% Clus5&6)−2.78*(% Clus2&4)>13 was the criteria for the high-risk recurrence.

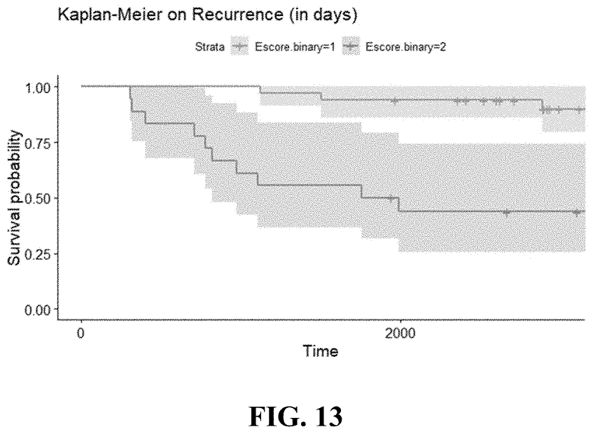

Referring now to FIG. 13, the disease-free-interval analysis using Kaplan Meier plots for the two group are shown. In these plots, binary categorization of the Escore results in clear separation of the survival curves (p=5E-05 with low scores being associated with marked decrease in likelihood of recurrence. In initial validation using the leave-one-out cross validation, Escore remained significantly associated with recurrence (p=0.006).

The biological heterogeneity in cancer is well recognized. This recognition has led to the understanding that not all cancers need to be treated aggressively. In the case of invasive breast cancer, gene expression assay-based trials such as the MINDACT (Microarray in the Determination of Adjuvant Chemotherapy) and TAILORx (Trial Assigning Individualized Options for treatment Rx) have documented that a significant number of women can safely avoid chemotherapy. Of note, both trials showed these assays provided limited discrimination power for patients in whom there was a disagreement between the clinical and molecular risk strata (i.e. Low clinical and high molecular or High clinical and low molecular). In spite of this, both assays were good at identifying classes of patients that the benefit from chemotherapy (high clinical and high molecular risk groups) and that can safely avoid chemotherapy (low clinical and low molecular risk groups).

Epidemiological studies have documented that overall survival rates for DCIS are greater than 95% at 10 years. It is natural to seek to identify categories of patients for whom therapy can be reduced. Additionally, there is concern about ‘overdiagnosis’ and hence overtreatment screen detected DCIS. DCIS has been traditionally treated with surgery followed by hormonal therapy and or radiotherapy to the breast to prevent recurrence of DCIS or development of invasive cancer. The current clinical trials (LORIS, LORD and COMET) are enrolling patients on histological features; this in part due to lack of good molecular markers. One of the major limitations of the IHC or mRNA panels is the amount of tissue required for analysis. This is particularly true in cases where important management decisions are going to be made on tiny fragment of “tumor” tissue in needle core biopsies. To minimize the tissue requirements, multiplex immunofluorescence (CELL DIVE™) was utilized to identify parameters associated with recurrence.

The instant disclosure is based on analysis of a single section of the tissue microarray (TMA) from patients with DCIS. Thirty-three markers were analyzed on a single paraffin section using 15 cycling rounds of staining and imaging. This is a major strength of the study. However, the analysis also resulted in dramatic loss of number of samples analyzed. A significant part of the loss was due to the requirement that field be composed almost entirely of DCIS cells. This criterion was used to reduce the impact of normal (contaminating) epithelial elements and made it easier to analyze the data as it did not require cell-level classification of the lesions. Further sophistication of the analysis algorithms is necessary to prevent such major losses.

In univariate analysis, only ER, PR, and HER2 were associated with likelihood of recurrence. This is consistent with prior literature and suggests that the result observed herein can be potentially generalizable. None of the other markers analyzed were associated with recurrence. Without bound by any theory, this is likely due to the fact that the current study did not have the power to detect additional prognostic role of features which have a weaker influence on outcome. For example, in contrast to the prior findings, the expression of both KI67 and COX2 was not found herein to be associated with recurrence in the context of DCIS. Moreover, mRNAs of the proliferation related genes play an important role in Oncotype Dx DCIS score.

Although the expression of SLC7A5 and cMET was not significant in univariate analysis, in the cluster algorithm, high expression of cMET (ER^{low}, HER2^{low}, SLC7A5^{low}; cluster 4) was associated low likelihood for recurrence. However, the presence of HER2+ status, trumped cMET expression and resulted in increased risk of recurrence. Of note, cMET and SLC7A5 have not been previously implicated in prognostication of DCIS. In invasive cancer, cMET overexpression is seen in metastatic tumors. SLC7A5 has described as a component of the MAMMASTRAT™ signature for ER+ stage II breast cancers, and more recently shown to be a key therapeutic target in ER+ breast cancer.

The combination of expression scores of ER/HER2/cMET and SLC7A5 markers contributed to development of the Escore algorithm. Escore was significantly predictive of likelihood of recurrence (p=0.00005). In preliminary validation using leave one out cross-validation (LOOCV) method, Escore remained significant (p=0.006). The results of the current analyses need to be validated in additional cohorts to understand the importance of the Escore. Further analyses will include replication of the algorithm using CELL DIVE™ that necessitates use of multiple markers for cell segmentation as well simpler methods using (just) the four markers. Success in generating the Escore using simple(r) IHC methods or hyperspectral imaging of 4-8 markers could result in rapid dissemination of the results and their implementation in clinical practice. Escore has the potential of identifying women with DCIS who could be spared additional therapies.

A recent database update resulted in reclassification of recurrence status of 4 patients from no-recurrence to recurrence. As these recurrences occurred beyond the late (9.6 yrs to 16.2 yrs) after initial diagnosis; these might be potentially new diseases. Only two of these 4 patients were in the better outcome group by E-score.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

What is claimed is:

1. A method of detecting one or more epithelial markers in a person suspected of having recurrence of ductal carcinoma in situ, the method comprising
providing a sample of blood, cells, or tissue from the person suspected of having recurrence of ductal carcinoma in situ; and
detecting one or more epithelial markers in the sample, wherein the one or more epithelial markers comprise estrogen receptor (ER), human epidermal growth factor receptor 2 (HER2), SLC7A5 and/or cMET.

2. The method of claim 1 where the person has low risk of recurrence of ductal carcinoma in situ when the expression of ER and/or cMET is high in the sample.

3. The method of claim 1 where the person has increased risk of recurrence of ductal carcinoma in situ when the expression of HER2 and/or SLC7A5 is high in the sample.

4. A method of detecting one or more epithelial markers in a person suspected of having invasive carcinoma, the method comprising
providing a sample of blood, cells, or tissue from a person suspected of having ductal carcinoma in situ; and
detecting one or more epithelial markers in the sample, where the one or more epithelial markers comprise estrogen receptor (ER), human epidermal growth factor receptor 2 (HER2), SLC7A5 and/or cMET.

5. The method of claim 4 where the person has low risk of having invasive carcinoma when the expression of ER and/or cMET is high in the sample.

6. The method of claim 4 where the person has increased risk of having invasive carcinoma when the expression of HER2 and/or SLC7A5 is high in the sample.

7. A method of treating recurrence of ductal carcinoma in situ, the method comprising
detecting increased expression of one or more epithelial markers in a sample of a subject suspected of having ductal carcinoma in situ, wherein the one or more epithelial markers comprise HER2 and/or SLC7A5;
providing to the subject at least one treatment regimen comprising chemotherapy, radiation therapy, endocrine therapy, breast-conserving surgery (lumpectomy), breast-removing surgery (mastectomy), and/or at least one therapeutically effective dose of a compound that inhibits the epithelial expression of HER2 and/or SLC7A5.

8. The method of claim 7 where the compound that inhibits the epithelial expression of HER2 and/or SLC7A5 comprises a pharmaceutically acceptable salt or a metabolite thereof.

9. The method of claim 7 where the subject is diagnosed with ductal carcinoma in situ.

10. The method of claim 7 where the subject comprises an animal, a human, a cell, and/or a tissue.

11.-14. (canceled)

15. The method of claim 4 where the invasive carcinoma comprises invasive ductal carcinoma (IDC), infiltrating ductal carcinoma, invasive lobular carcinoma (ILC), adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma, and/or mixed carcinoma having features of both invasive ductal and lobular.

16. (canceled)

17. The method of claim 5 where low risk is define by escore analysis.
